(19) United States
(12) Patent Application Publication
(10) Pub. No.: US 2005/0228273 A1
(43) Pub. Date: Oct. 13, 2005

(54) Radiographic imaging apparatus, radiographic imaging program and information recording medium

(75) Inventor: Yasuaki Tamakoshi, Tokyo (JP)

Correspondence Address: MUSERLIAN, LUCAS AND MERCANTI, LLP, 475 PARK AVENUE SOUTH, 15TH FLOOR, NEW YORK, NY, 10016, US

(73) Assignee: Konica Minolta Medical & Graphic, Inc.

(21) Appl. No.: 11/098,118

(22) Filed: Apr. 4, 2005

(30) Foreign Application Priority Data
Apr. 7, 2004 (JP) 2004-113628

(51) Int. Cl. H05G 1/64; A61B 5/05

(52) U.S. Cl. 600/425

(57) ABSTRACT

A radiographic imaging apparatus includes: a plane detection device to obtain radiographic image; an injection device communication unit to perform communication with an injection device which automatically injects a contrast agent; an irradiation device communication unit to perform communication with an irradiation device; an input unit; a control unit including a plurality of control modes including a linking mode of allowing linking among a timing contrast agent injection, a timing of obtaining a radiographic image and a timing of the irradiation, the control unit controlling the injection device, plane detection device and irradiation device in a control mode selected from the plurality of control modes; and a judgment unit to judge whether control in the linking mode by the control unit is possible, wherein selection of the linking mode is possible only when the judgment unit has judged that the linking mode is possible.

### BACKGROUND OF THE INVENTION

1. Field of the Invention

The present invention relates to a radiographic imaging apparatus, a radiographic imaging program and an information recording medium, and particularly to a radiographic imaging apparatus, a radiographic imaging program and an information recording medium for imaging using a contrast agent.

2. Description of the Related Art

Radiographic images as typified by X-ray images have been widely used for sick diagnosis. For imaging apparatuses to take radiographic images, there have been known apparatuses which use, as a detection unit to detect radiation that has passed through the subject, a radiographic film or a photostimulable-phosphor sheet which obtains a visible image by utilizing storage phosphor (photostimulable-phosphor) that accumulates radiated radiation energy.

In recent years, there have been sold radiographic imaging apparatuses which include a so-called FPD (Flat Panel Detector), which is a kind of plane detection device, for outputting radiographic image data showing a radiographic image detected by a plurality of solid-state photodetectors arranged two-dimensionally.

Moreover, upon radiographic imaging, to clearly show the tissue having a small difference in radiation absorption rate with respect to the surrounding tissue, radiographic imaging using a contrast agent has been performed. Examples of the radiographic imaging using a contrast agent include: X-ray gastrointestinal tract radiography for imaging the digestive tract such as stomach and intestines by using a barium sulfate contrast agent taken by drinking or an enema; X-ray lymphography for imaging lymph vessels by using a contrast agent injected into the lymph vessels; X-ray imaging for imaging the urinary tract, uterus and oviduct, joints and the like by using a contrast agent injected thereinto; and X-ray angiography for imaging vessels by using a contrast agent injected thereinto.

Major examples of X-ray imaging using a contrast agent injected into the vessels include: CT imaging using a contrast agent injected into the veins; intravenous pyelography for imaging excretory organs upon discharge of the contrast agent injected into the veins; and imaging of target tissue performed by inserting the top end of the catheter in an appropriate position, allowing the top end to move to the vicinity of the target tissue, and injecting a contrast agent in the vicinity of the target tissue.

Meanwhile, there has been disclosed that an X-ray tomography apparatus for taking a tomogram of a subject, which includes built-in X-ray tube and X-ray detection unit, may include a built-in contrast agent injection device and that a plane detection device may be used as a radiation detection unit (for example, see JP-10-295680-A). In such an imaging apparatus for taking a tomogram, an expensive drive unit for permitting relative movement between the X-ray tube and the X-ray detection unit with high accuracy is required. Therefore, the apparatus is naturally an all-in-one apparatus also including a built-in contrast agent injection device having high durability and reliability of durability. Moreover, there has also been disclosed that a CT scanner and a drug solution injection device are separate devices which are linked with each other (for example, see JP-2004-298610-A). However, the CT scanner and the drug solution injection device are always linked with each other, and it is not described that detection as to whether or not the linking is possible is performed.

As a result of keen study on this, the inventor has found that it is preferred that the irradiation device such as an X-ray tube, radiographic imaging apparatus including an X-ray detection unit such as a plane detection device, and contrast agent injection device are separate devices, unlike the expensive apparatus of all-in-one type as disclosed in JP-10-295680-A.

That is, such an all-in-one type radiographic imaging apparatus including a built-in contrast agent injection device tends to be expensive, and therefore, it is difficult for a small-scale medical institution to introduce such an apparatus. In addition, if radiographic imaging using a contrast agent cannot be performed unless such an expensive apparatus is introduced, the cost of radiographic imaging using a contrast agent is increased, and a burden on the patients becomes large due to high medical cost.

Thus, it can be thought that an irradiation device, a plane detection device and a contrast agent injection device are configured to be separate devices, and that radiographic imaging using a contrast agent is performed by combining these devices. Such combination of the separate devices makes it possible to freely utilizes each device by, for example, combining only the irradiation device and the plane detection device when the contrast agent is not used for imaging, and using each device for another purpose by combining each device with another device. Accordingly, as compared with the all-in-one type apparatus as a single body including the irradiation device, plane detection device and contrast agent injection device, applicability of each device is increased. Moreover, frequency in use of each device increases, and therefore, the devices can be efficiently used. Thus, the cost per imaging can be reduced.

This is because, if the devices are separate single bodies, each device can be used for another purpose when combined with another device. As compared with a dedicated apparatus of all-in-one type, the applicability of each device is increased, and the utilization ratio of each device is increased since the devices are not kept idle. As a result, the cost per imaging can be reduced.

Further, the applicability and frequency in use differ among the irradiation device, plane detection device and contrast agent injection device, depending on the institution and hospital. In some institution or hospital, two irradiation devices, three detection devices and two contrast agent injection devices may be combined. In another institution or hospital, one irradiation device, one detection device and one contrast agent injection device may be combined. In this way, these devices can be freely combined in each institution or hospital.

In addition, when there are a plurality of devices of one kind in an institution or hospital, it is preferred that any combination among the devices is possible for imaging using a contrast agent.

However, for example, there is a case where it is preferred that the contrast agent injection device, irradiation device and plane detection device are linked, such as when imaging is performed by injecting a contrast agent into the vessels. Thus, it has been found that there is a problem when the devices as single bodies are merely combined, as to how to make the linking among these devices reliable.

That is, since the contrast agent injection device, irradiation device, and plane detection device are separate devices, when the linking controlling side tries controlling of the linking even in a state where the linking between the linking controlling side and the linking controlled side is not possible, control of making the linking performed is started even though the linking cannot be performed. Thus, it is found that there are problems that inappropriate radiographic imaging is performed, inappropriate contrast agent injection is performed, and the injection is stopped in the middle.

### SUMMARY OF THE INVENTION

The present invention is made to solve the above-described problems. An object of the invention is to provide: a radiographic imaging apparatus capable of realizing, with reliability and less cost, radiography using a contrast agent in a linking mode where an irradiation device, an injection device and a plane detection device are combined to be linked with each other, each device being a single body separate from each other and permitted to be used for another purpose by being combined with another device, and realizing radiography in a control mode other than the linking mode even when radiographic imaging in the linking mode is not possible, thus realizing high efficiency in use of the apparatus in total; a radiographic imaging program for allowing the apparatus to operate; and an information storage medium storing the program.

In order to attain the above objects, in accordance with a first aspect of the present invention, a radiographic imaging apparatus comprises: a plane detection device to obtain radiographic image information by using a plurality of solid-state photodetectors arranged two-dimensionally; an injection device communication unit to perform communication with an injection device which automatically injects a contrast agent; an irradiation device communication unit to perform communication with an irradiation device which performs irradiation; an input unit; a control unit which includes a plurality of control modes including a linking mode of allowing, by using the injection device communication unit and the irradiation device communication unit, linking among a timing of injecting the contrast agent by the injection device, a timing of obtaining a radiographic image from the plane detection device and a timing of performing the irradiation by the irradiation device, the control unit controlling the injection device, the plane detection device and the irradiation device in a control mode selected from the plurality of control modes in accordance with the input performed through the input unit; and a judgment unit to judge whether or not control in the linking mode by the control unit is possible, wherein selection of the linking mode as the control mode is possible only when the judgment unit has judged that the control in the linking mode is possible.

Thus, according to the first aspect of the invention, when the judgment unit has judged that the control in the linking mode by the control unit is possible, it is possible for the control unit to perform control in the linking mode of allowing linking among the timing of injecting the contrast agent by the injection device, the timing of obtaining a radiographic image from the plane detection device and the timing of performing the irradiation by the irradiation device, through the communication with the irradiation device and the injection device. Therefore, while reliability in radiography in the linking mode using a contrast agent is secured, it is possible to freely combine for another purpose the injection device, irradiation device and plane detection device, which are single bodies separate from each other. Thus, it is not necessary to use an expensive apparatus of all-in-one type including the irradiation device, injection device and plane detection device, thereby realizing inexpensive radiography using a contrast agent. Moreover, a control mode other than the linking mode is possible even when radiography in the linking mode is not possible. Therefore, efficiency in use of the radiographic imaging apparatus is increased in total.

It is preferred that selection of a control mode other than the linking mode is also possible, and, when the control mode other than the linking mode is selected, the control unit does not link at least one of the timing of injecting the contrast agent by the injection device, the timing of obtaining the radiographic image from the plane detection device and the timing of performing the irradiation by the irradiation device, with the others.

Thus, selection of a control mode other than the linking mode is also possible even when the judgment unit has judged that the control in the linking mode by the control unit is not possible. Therefore, it is possible to allow any one of the devices to cooperate with another device freely, or to use any one of the devices as it is.

It is preferred that detection as to whether or not the communication between the irradiation device communication unit and the irradiation device is possible is performed, and the judgment unit judges whether or not the control in the linking mode by the control unit is possible based on a result of the detection.

Thus, the judgment unit judges whether or not the control in the linking mode by the control unit is possible based on a result of the detection as to whether or not the communication between the irradiation device communication unit and the irradiation device is possible. Therefore, reliability in radiography in the linking mode using a contrast agent is increased.

It is preferred that detection as to whether or not irradiation toward the plane detection device by the irradiation device is possible is performed, and the judgment unit judges whether or not the control in the linking mode by the control unit is possible based on a result of the detection.

Thus, the judgment unit judges whether or not the control in the linking mode by the control unit is possible based on a result of the detection as to whether or not irradiation toward the plane detection device by the irradiation device is possible. Therefore, reliability in radiography in the linking mode using a contrast agent is increased.

It is preferred that detection as to whether or not the communication between the injection device communication unit and the injection device is possible is performed, and the judgment unit judges whether or not the control in the linking mode by the control unit is possible based on a result of the detection.

Thus, the judgment unit judges whether or not the control in the linking mode by the control unit is possible based on a result of the detection as to whether or not the communication between the injection device communication unit and the injection device is possible. Therefore, reliability in radiography in the linking mode using a contrast agent is increased.

It is preferred that the control unit is permitted to control the plane detection device through wireless communication, the apparatus further comprises a wireless communication state detection unit to detect a state of the wireless communication, and the judgment unit judges whether or not the control in the linking mode by the control unit is possible based on a result of the detection by the wireless communication state detection unit.

Thus, the control unit can control the plane detection device through wireless communication. Therefore, it is not necessary to install a cable or the like, and there is degree of freedom in disposing the plane detection device. Moreover, the plane detection device is easily attached and detached, and whether or not the control in the linking mode is possible is judged based on the result of the detection on the state of wireless communication with the plane detection device. Therefore, it is possible to reduce adverse effect due to undependability in wireless communication, and increase reliability in radiography in the linking mode using a contrast agent.

It is preferred that detection as to whether or not automatic injection of the contrast agent by the injection device is possible is performed, and the judgment unit judges whether or not the control in the linking mode by the control unit is possible based on a result of the detection.

Thus, the judgment unit judges whether or not the control in the linking mode by the control unit is possible based on a result of the detection as to whether or not automatic injection of the contrast agent by the injection device is possible. Therefore, reliability in radiography in the linking mode using a contrast agent is increased.

It is preferred that the apparatus further comprises an image processing unit to perform image processing on a radiographic image obtained by the plane detection device, wherein, when the linking mode is selected, the control unit controls the image processing unit to perform the image processing based on an image processing condition in accordance with the linking mode.

Thus, when the linking mode is selected, the control unit controls the image processing unit to perform the image processing based on an image processing condition in accordance with the linking mode. Therefore, it is possible to obtain an image with high diagnostic ability through image processing by the image processing unit based on the image processing condition set in accordance with the linking mode.

It is preferred that, when a control mode other than the linking mode is selected, the control unit controls the image processing unit to perform the image processing based on an image processing condition in accordance with the selected control mode other than the linking mode.

Thus, when the linking mode is not selected, the control unit controls the image processing unit to perform the image processing based on an image processing condition in accordance with a control mode other than the linking mode. Therefore, it is possible to obtain a more appropriate image through image processing by the image processing unit based on the image processing condition in accordance with the control mode other than the linking mode.

It is preferred that, when the linking mode is selected, the irradiation device communication unit performs the communication to set a condition for controlling the irradiation device in accordance with the linking mode.

Thus, when the linking mode is selected, the irradiation device communication unit performs the communication to set a condition for controlling the irradiation device in accordance with the linking mode. Therefore, it is possible to obtain a more appropriate image through irradiation based on the condition appropriate for controlling the irradiation device in accordance with the linking mode.

It is preferred that, when a control mode other than the linking mode is selected, the irradiation device communication unit performs the communication to set a condition for controlling the irradiation device in accordance with the selected control mode other than the linking mode.

Thus, when a control mode other than the linking mode is selected, the irradiation device communication unit performs the communication to set a condition for controlling the irradiation device in accordance with the selected control mode other than the linking mode. Therefore, it is possible to obtain a more appropriate image through irradiation based on the condition appropriate for controlling the irradiation device in accordance with the control mode other than the linking mode.

It is preferred that, when the linking mode is selected, the irradiation device communication unit performs the communication so that the irradiation device performs the irradiation after a predetermined time period from the timing of injecting the contrast agent by the injection device.

Thus, when the linking mode is selected, the irradiation device communication unit performs the communication so that the irradiation device performs the irradiation after a predetermined time period from the timing of injecting the contrast agent by the injection device. Therefore, the irradiation can be performed in a more appropriate timing, and thus it is possible to obtain a more appropriate radiographic image.

It is preferred that, when the linking mode is selected, the control unit controls the plane detection device to read a radiographic image from the plane detection device after a predetermined irradiation reading time period from the timing of performing the irradiation by the irradiation device.

Thus, when the linking mode is selected, the control unit controls the plane detection device to read a radiographic image from the plane detection device after a predetermined irradiation reading time period from the timing of performing the irradiation by the irradiation device. Therefore, the radiographic image can be obtained in a more appropriate timing, and thus it is possible to obtain a more appropriate radiographic image.

It is preferred that the control unit is permitted to control the plane detection unit through wireless communication, and the control unit performs wireless transmission of information concerning a timing of reading the radiographic image, the information being transmitted to the plane detection device prior to radiographic imaging.

For example, with a unit which transmits a read signal in a timing of reading a radiographic image obtained after irradiation, it is not possible to read the radiographic image in an appropriate timing if the communication in the timing of reading the radiographic image is failed. However, in the invention, information concerning a timing of reading the radiographic image is transmitted to the plane detection device prior to radiographic imaging. Therefore, even when the transmission is failed, by performing transmission again or the like, the radiographic image can be read in an appropriate timing, and thus it is possible to obtain a more appropriate image.

It is preferred that the apparatus further comprises an operation screen displaying unit to display an operation screen allowing selection of the linking mode when the judgment unit has judged that the control in the linking mode is possible, and display an operation screen not allowing selection of the linking mode when the judgment unit has judged that the control in the linking mode is not possible.

Thus, the operator can recognize whether or not selection of the linking mode is possible based on the display on the operation screen, and an appropriate control mode can be selected based on the availability of the selection.

It is preferred the apparatus further comprises an operation screen displaying unit to display an operation screen showing an option for selection of the linking mode, wherein, in a case where the selection of the linking mode is performed through the input unit, the selection of the linking mode is made when the judgment unit has judged that the control in the linking mode is possible, and the selection of the linking mode is not made when the judgment unit has judged that the control in the linking mode is not possible, by displaying on the operation screen that the selection of the linking mode is not possible.

Thus, based on the display on the operation screen, the operator can recognize control modes including the linking mode and select a desired control mode. In addition, the operator can recognize all kinds of control modes that are originally included. Since information that the selection of the linking mode is not possible is displayed on the operation screen when the linking mode is selected even though the control in the linking mode is not possible, the operator can recognize whether the selection of the linking mode is possible, and select an appropriate control mode based on the availability of the selection.

It is preferred that the plane detection device is attachable and detachable, when the plane detection device is attached, the plane detection device is adapted to be attached to a radiographing device such that the plane detection device is in a position toward which the irradiation by the irradiation device is performed, the irradiation device is accommodated in the radiographing device.

Thus, when the plane detection device is attached to the radiographing device, the plane detection device is in a position toward which the irradiation by the irradiation device is performed. Therefore, reliability in radiography in the linking mode using a contrast agent is increased.

It is preferred that the plane detection device is a portable FPD of a cassette type.

Thus, the plane detection device is easily attached and detached, and therefore, the degree of freedom in radiography is increased.

In accordance with a second aspect of the invention, a radiographic imaging program allows a computer connected to and capable of communicating with a plane detection device to obtain radiographic image information, an injection device to perform automatic injection of a contrast agent, and an irradiation device to perform irradiation, to realize: a control function of controlling the injection device, the plane detection device and the irradiation device in a control mode selected from a plurality of control modes in accordance with input performed through an input unit, the plurality of control modes including an linking mode of allowing linking among a timing of injecting the contrast agent by the injection device, a timing of obtaining a radiographic image from the plane detection device and a timing of performing the irradiation by the irradiation device; a judgment function of judging whether or not control in the linking mode is possible; and a selection possibility determining function of allowing selection of the linking mode as the control mode only when the judgment function has judged that the control in the linking mode is possible.

In accordance with a third aspect of the invention, a computer-readable information storage medium stores the radiographic imaging program.

### BRIEF DESCRIPTION OF THE DRAWINGS

Although the present invention will become more fully understood from the detailed description given hereinbelow and the accompanying drawings which are given by way of illustration only, and thus are not intended as a definition of the limits of the present invention, and wherein;

FIG. 1 is a diagram showing an example of a schematic configuration of an X-ray imaging apparatus, an injection device and an X-ray source which are connected to the X-ray imaging apparatus, according to the embodiment;

FIG. 2 is a view showing a schematic configuration of a radiographing device having a C-shape arm, which is applicable to the embodiment;

FIG. 3 is a perspective view showing a structure of a main part of the injection device of the embodiment;

FIG. 4 is a view showing an example of a selection screen of an input unit of the X-ray imaging apparatus of the embodiment;

FIG. 5 is a flowchart showing an example of processing of judging whether selection of a linking mode is possible or not, the processing being performed by the X-ray imaging apparatus of the embodiment; and

FIG. 6 is a flowchart showing an example of processing of judging whether control in the linking mode is performed or not, the processing being performed by the X-ray imaging apparatus of the embodiment.

### DESCRIPTION OF THE PREFERRED EMBODIMENT

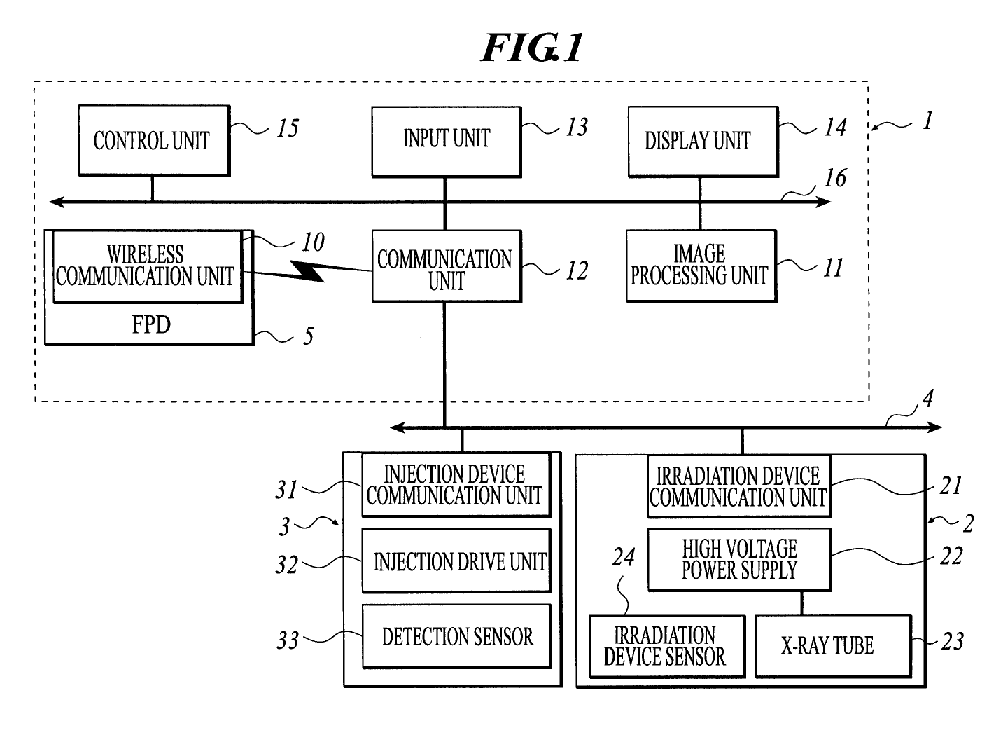

Hereinbelow, with reference to FIGS. 1 to 6, an X-ray imaging apparatus which is an embodiment of the radiographic imaging apparatus according to the present invention will be described. Note that the description below is not intended to limit the technical scope of the claims and definitions of the terms thereof.

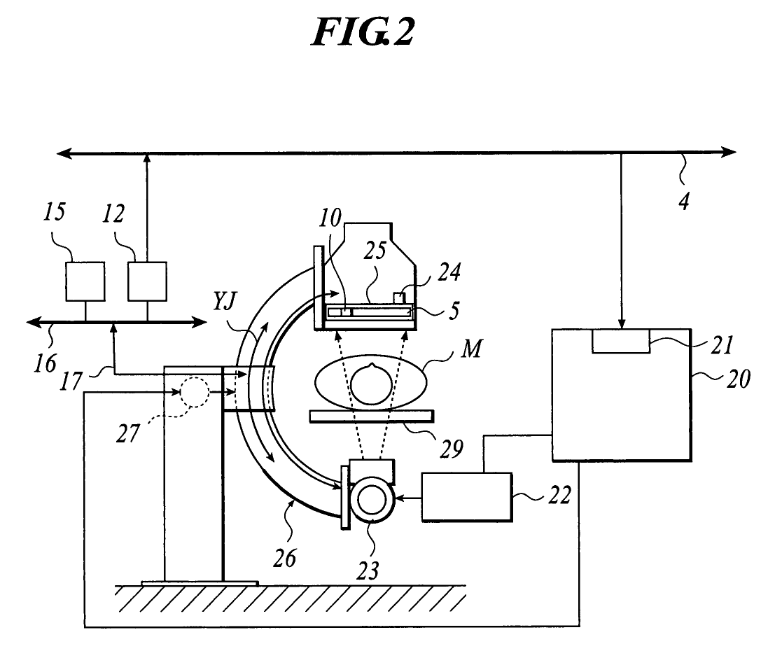

In this embodiment, the radiographic imaging apparatus is an X-ray imaging apparatus 1 to radiate X-rays as a radiation. As shown in FIG. 1, an X-ray source 2 which is a kind of irradiation device to irradiate a subject M (see FIG. 2) and an injection device 3 to automatically inject a contrast agent into the body of the subject M are configured to be connectable to the X-ray imaging apparatus 1 through a network 4. In the embodiment described below, X-rays are used as a radiation; however, the radiations applicable to the radiographic imaging apparatus according to the present invention are not limited to X-rays.

The X-ray imaging apparatus 1 includes a flat panel detector (hereinafter referred to as “FPD”) 5 as a plane detection device to read X-rays radiated from the X-ray source 2 and obtain X-ray image information. The X-ray imaging apparatus 1 is capable of detecting X-rays which have radiated from the X-ray source 2 and passed through the subject M. Here, the network 4 may be a communication line exclusive to the above system; however, the network 4 is preferably an existing line such as a wireless LAN or Ethernet (registered trademark), since, for example, otherwise a degree of freedom for the system configuration is reduced. Additionally, to the network 4, there may be connected a server to manage information on X-ray imaging such as information on the subject M etc. and imaging conditions. The X-ray imaging apparatus 1, X-ray source 2 and injection device 3 can also be used individually, and may be connected to and disconnected from the network 4 as necessary.

The X-ray source 2 includes an X-ray tube 23 to radiate X-rays, and a high voltage power supply 22 to supply a high voltage to the X-ray tube 23. When a high voltage is supplied from the high voltage power supply 22 to the X-ray tube 23, the X-ray source 2 generates X-rays and irradiates the subject M with the generated X-rays. A limiting device (not shown) to limit X-rays to be radiated from the X-ray tube 23 to a desired field is provided to the X-ray source 2. The limiting device limits the X-rays in accordance with the type of imaging, the target for imaging, and the like.

The X-ray source 2 includes an irradiation device communication unit 21 to communicate with external devices such as the X-ray imaging apparatus 1. Irradiation conditions such as an X-ray dose and an X-ray limitation amount, which are inputted from external devices through the irradiation device communication unit 21, are transmitted to the X-ray source 2 as electric signals. In accordance with the irradiation conditions inputted from the external devices, a predetermined tube potential is applied to the X-ray tube 23 to apply a tube current, and the X-rays are limited to a predetermined field by the limiting device. In this way, X-rays of an appropriate dose for imaging are radiated to the subject.

Moreover, an irradiation device sensor 24 to detect whether it is possible to radiate X-rays to the FPD 5 normally is provided to the X-ray source 2. The irradiation device sensor 24 transmits as a signal a detection result from the irradiation device communication unit 21 to the X-ray imaging apparatus 1.

For example, as a state where it is not possible for the X-ray source 2 to radiate X-rays to the FPD 5, the state being detected by the irradiation device sensor 24, there is a state where the high voltage power supply 22 or the X-ray tube 23 of the X-ray source 2 is out of order or deteriorated, a state where the high voltage power supply 22 is not ready and thus cannot supply a high tension power immediately, and the like. Even when there is no problem with the high voltage power supply 22 and the X-ray tube 23 per se, when the X-ray source 2 is not linked with the FPD 5 used for imaging or when the X-ray radiation direction of the X-ray source 2 is not oriented to the FPD 5, the irradiation device sensor 24 may detect such states as a state where X-rays cannot be radiated to the FPD 5 normally.

The irradiation device sensor 24 may be, for example, a sensor to measure the dose of X-rays radiated from the X-ray tube 23 and detect whether or not a predetermined dose of X-rays are radiated under predetermined conditions including a tube potential and a tube current supplied to the X-ray tube. Further, an operation unit for adjusting the X-ray radiation direction of the X-ray source 2 may be provided, and the irradiation device sensor 24 may detect that predetermined adjustment through operation of the operation unit has been completed. Furthermore, the irradiation device sensor 24 may be a sensor to detect whether or not the X-ray radiation direction of the X-ray source 2 is oriented to the FPD 5. Moreover, pre-irradiation with a predetermined radioactive ray may be performed by the irradiation device 2, and the irradiation device sensor 24 may detect whether or not the predetermined radioactive ray has been detected by the FPD 5. In a case of, for example, a radiographing device as shown in FIG. 2 in which the X-ray source 2 is fixedly provided in a predetermined position, a C-shape arm 26 used for radiating a radioactive ray in a predetermined direction is provided, and a loading slot 25 for setting the FPD 5 in a predetermined position is provided, the irradiation device sensor 24 may be a sensor to detect whether or not predetermined loading of the FPD 5 in the slot 25 has been performed.

In this case, for example, the FPD 5 is of a portable type such as a cassette type FPD, and attachable to the loading slot 25 and detachable therefrom to be freely carried. It is preferred that a signal wire 17 of the radiographic imaging apparatus 1 is provided along the C-shape arm 26, and that the end of the signal wire 17 is provided to the loading slot 25, so that the radiographic imaging apparatus 1 is capable of performing wireless communication with a wireless communication unit 10 of the FPD 5, thus enhancing the certainty of the wireless communication therebetween. When the loading slot 25 is loaded with the FPD 5, wired connection may be established between the radiographic imaging apparatus 1 and the FPD 5. The C-shape arm 26 enables X-ray imaging of the subject M on a bed 29 from an arbitrary direction, by being rotated in YJ direction of FIG. 2 by an arm drive unit 27 controlled by the control unit 20 of the X-ray source 2.

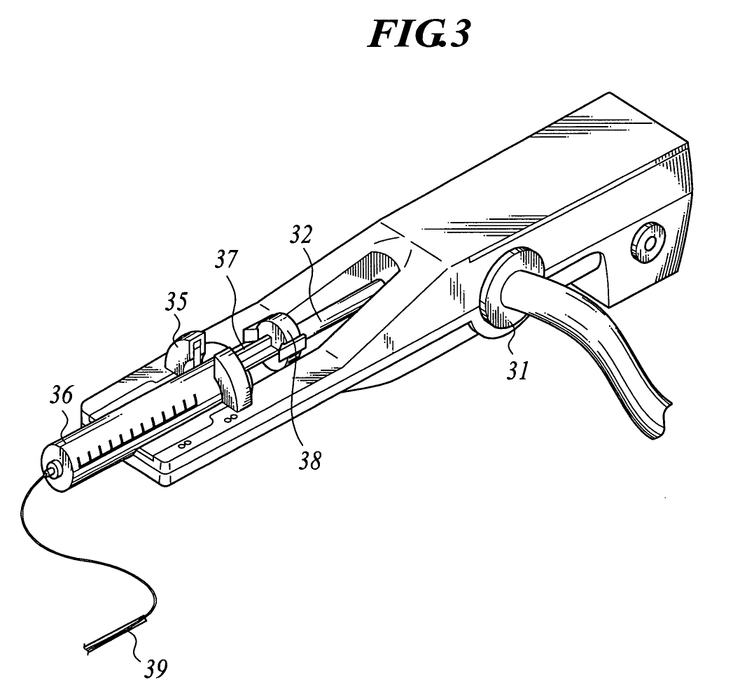

As shown in FIG. 3, the injection device 3 includes a holding unit 35 to hold a syringe 36 which contains a contrast agent, and a catheter 39 connected to the syringe 36 is inserted into the body of the subject M. The injection device 3 includes an injection drive unit 32 to grip a plunger 37 of the syringe 36 by a chuck 38 provided at an end thereof, and sends the contrast agent inside the syringe 36 into the body of the subject M through the catheter 39.

The injection device 3 includes an injection device communication unit 31 to communicate with external devices such as the X-ray imaging apparatus 1. The injection drive unit 32 is capable of adjusting the amount of the contrast agent to be injected from the syringe 36, the injection speed thereof, and the like. The injection drive unit 32 adjusts the injection amount of the contrast agent, injection speed thereof, and the like in accordance with injection conditions inputted from the external devices through the injection device communication unit 31.

Moreover, the injection device 3 includes a detection sensor 33 as a detection unit to detect whether it is possible to inject the contrast agent normally. The detection sensor 33 transmits a detection result as a signal to the X-ray imaging apparatus 1 from the injection device communication unit 31.

Examples of states where it is not possible for the injection device 3 to inject the contrast agent normally include a case where the injection device 3 is out of order, a case where the syringe 36 is not loaded in a predetermined position of the injection device 3, a case where the amount of the contrast agent remained in the syringe 36 is not sufficient for injection operation, and the like. Additionally, in a case where a contrast agent injection end, for example, the catheter 39 or a needle of a contrast agent injector, is not inserted in the body of the subject M normally, such as when the contrast agent injection end is deviated from the contrast agent passage, in a case where the contrast agent is leaking in the midway through the contrast agent passage, and in a case where the contrast agent passage is clogged, it is not possible to inject the contrast agent normally.

The detection sensor 33 may be, for example, a sensor to detect whether the syringe 36 is loaded normally. Moreover, the detection sensor 33 may be a sensor to detect the amount of the contrast agent remained in the syringe 36. A sensor to detect the driving force of the injection drive unit 32 at the time of injecting the contrast agent into the injection device 3 may be provided as the detection sensor 33, and the sensor may detect whether or not the driving force for outputting the contrast agent is within a predetermined range. In this case, for example, when the contrast agent injection end is not inserted in the body of the subject M normally or when the contrast agent passage is clogged, the driving force is above the predetermined range, and when the contrast agent is leaking in the midway through the contrast agent passage, the driving force is below the predetermined range. Therefore, by detecting such a driving force, it is possible to judge whether or not the injection device 3 is in a state of being able to inject the contrast agent normally. Further, an input unit (not shown) may be provided, and the detection unit 33 may be a sensor to detect whether or not an input through the operation unit is performed to show that it is confirmed that the injection device 3 is in a state of being able to automatically inject the contrast agent. Furthermore, for example, a pressure sensor to measure a pressure may be provided as the detection sensor 33 at a portion in the catheter 39, and whether the contrast agent is injected with a predetermined pressure may be detected.

The FPD 5 provided in the X-ray imaging apparatus 1 is, for example, an FPD of a cassette type. The FPD 5 is disposed in a position which is within the irradiation field of the X-rays radiated from the X-ray source 2 and at which X-rays that have radiated from the X-ray source 2 and passed through the subject M can be detected. With such cassette type FPD 5, the FPD 5 can be easily exchanged. Note that the FPD 5 is not limited to a cassette type FPD.

The FPD 5 includes the wireless communication unit 10 to perform communication with such as a control unit 15 (to be described later) of the X-ray imaging apparatus 1 by using a wireless signal. An X-ray image from the FPD 5 is read through the wireless communication unit 10 in accordance with reading conditions inputted from the external devices. Note that the communication performed by the wireless communication unit 10 is not limited to communication using a wireless signal. Prior to imaging, information such as a timing of reading X-ray image information is sent from the control unit 15 to the FPD 5. The FPD 5 reads the X-ray image information based on such information.

Optical communication using light such as infrared rays, visible light, or ultraviolet rays, communication using an electric wave, and the like may be given as examples of the wireless communication. The data amount of radiographic image data is usually 1 MB or more, which is a large data amount; however, it is desired that the radiographic image be checked as soon as the imaging is performed. Therefore, to transmit radiographic image data from the FPD 5, it is preferred that the communication is performed using an electric wave with a frequency of 300 MHz or more (in particular, 800 MHz or more) or through optical communication, since high-speed data communication is easily performed through such communication. Meanwhile, for control in a linking mode (to be described later), it is required that timely communication is assured. Therefore, it is preferred that the communication is performed using an electric wave with a frequency of 500 MHz or less (in particular, 100 MHz or less) where communication cannot be easily influenced by obstacles in the way or a reflector. Accordingly, the communication for control in the linking mode and the communication for transmission of radiographic image data may be performed in different manners of communication.

The X-ray imaging apparatus 1 includes a wireless communication state detection unit to detect a communication state of the wireless communication unit 10. The control unit 15 is thus capable of detecting whether or not it is possible to appropriately transmit and receive information to and from the FPD 5 through the wireless communication unit 10.

In this embodiment, the wireless communication unit 10, a communication unit 12 (to be described later) or the like functions as the wireless communication state detection unit; however, the wireless communication state detection unit is not limited to thereto. For example, a wireless communication state detection unit may be provided in a device on the reception side of the wireless communication, and this wireless communication state detection unit may detect strength of the radio wave of the wireless communication. Moreover, a wireless communication state detection unit may be provided in another device on the reception side of the wireless communication, and this wireless communication state detection unit may detect an effective transfer rate of data to the device on the reception side. Thus, the communication state can be detected.

Moreover, the FPD 5 includes a function state detection unit (not shown) to detect whether or not the FPD 5 is in a state of being able to detect X-rays normally. Function state information which is a result of detection performed by the function state detection unit is transmitted as a wireless signal from the wireless communication unit 10 to the control unit 15 of the X-ray imaging apparatus 1.

Further, the X-ray imaging apparatus 1 includes an image processing unit 11 to perform various kinds of image processing, such as gradation processing, to the image information of an X-ray image obtained by the FPD 5.

Moreover, the X-ray imaging apparatus 1 includes the communication unit 12. When the X-ray imaging apparatus 1 is connected to the X-ray source 2 and the injection device 3 through the network 4, the communication unit serves as an irradiation device side communication unit to communicate with the irradiation device communication unit 21 of the X-ray source 2, and as an injection device side communication unit to communicate with the injection device communication unit 31 of the injection device 3. The X-ray source 2 and the injection device 3 are enabled to transmit and receive various information to and from the X-ray imaging apparatus 1. The communication unit 12 is also connected to the FPD 5 through a wireless signal, and the communication unit 12 transmits and receives various information to and from the wireless communication unit 10 of the FPD 5. In other words, the communication unit 12 is configured to transmit information such as reading conditions, e.g. timing of reading detected image information, to the FPD 5. The FPD 5 reads the image information based on the transmitted information. The image information detected and read by the FPD 5 is transmitted to the image processing unit 11 through the communication unit 12.

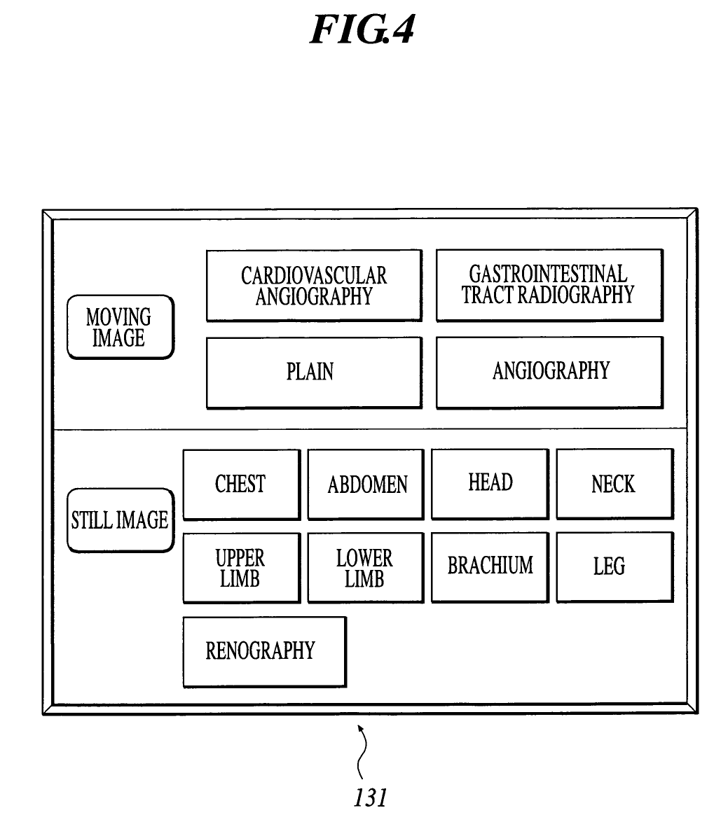

The X-ray imaging apparatus 1 includes an input unit 13 to input selection of an imaging region and an imaging mode, information on the subject M, conditions for imaging, instructions for image information reading and image information transmission and reception, and the like. The input unit 13 is, for example, as shown in FIG. 4, an operation panel 131 as an operation screen displaying unit. The user can input various information by operating the operation panel 131. More specifically, in this embodiment, as shown in FIG. 4, there is displayed a selection screen through which selection between moving image and still image, selection of imaging region, selection of kinds of imaging, and selection of whether or not the contrast agent is used in imaging can be performed. The user can easily set the imaging conditions and the like by selecting a desired imaging region and the like from among the options displayed on the selection screen.

Note that the input unit 13 is not limited to the operation panel 131, and may be any unit as long as it is possible to set various processing contents. For example, the input unit 13 may include a mouse and a keyboard so that various information is inputted by operating the mouse or pressing the keys of the keyboard. Further, the instructions and information which can be inputted from the input unit 13 are not limited to the above.

The X-ray imaging apparatus 1 includes a display unit 14. The display unit 14 includes, for example, a CRT (Cathode Ray Tube), an LCD (Liquid Crystal Display), or the like, and displays such as information inputted from the input unit 13 and image information sent from the FPD 5.

The X-ray imaging apparatus 1 includes the control unit 15 to control each unit. The control unit 15 includes, for example, a CPU (Central Processing Unit) (not shown), and is a computer communicably connected to the FPD 5, injection device 3, and X-ray source 2. The control unit 15 performs various processing in accordance with predetermined programs stored in a storage unit (not shown) such as a ROM (Read Only Memory), a computer-readable information storage medium, and other storage units.

In particular, in this embodiment, stored in the storage unit are radiographic imaging programs for allowing the control unit 15 to realize: a control function of controlling the injection device 3, FPD 5 and X-ray source 2 in a control mode selected from a plurality of control modes in accordance with input from the input unit, the plurality of control modes including a linking mode of allowing linking among the timing of injecting the contrast agent by the injection device 3, the timing of obtaining a radiographic image from the FPD 5, and the timing of X-ray radiation by the X-ray source 2; a judgment function of judging whether or not control in the linking mode is possible; and a selection possibility determining function of permitting selection of the linking mode as a control mode only when the judgment function has judged that the control in the linking mode is possible. The control unit 15 reads the radiographic imaging programs to develop them in a predetermined work area, and controls each unit of the device to perform necessary processing in accordance with the radiographic imaging programs.

The information inputted from the input unit 13 and the signal received by the communication unit 12 are sent to the control unit 15. The control unit 15 then functions as the judgment unit to judge whether imaging in the linking mode, in which the FPD 5, injection device 3 and X-ray source 2 are linked with each other and imaging is performed, is possible or not, based on the signal sent from the input unit 13 and the communication unit 12. That is, for example, the control unit 15 causes a predetermined signal to be transmitted from the communication unit 12 to each device, and judges whether or not each device is appropriately connected to each other and in a state of being able to communicate normally, based on whether a predetermined signal such as a reading possible signal, an injection possible signal and an irradiation possible signal is sent from the FPD 5, injection device 3 and X-ray source 2 in response to the signal transmitted from the communication unit 12. Moreover, the control unit 15 judges whether or not the injection device 3 and the X-ray source 2 can operate normally, based on the detection results of the detection units of the injection device 3 and the X-ray source 2 which are transmitted to the control unit 15 through the communication unit 12. Further, the control unit 15 judges based on these judgment results whether or not it is possible to perform imaging in the linking mode where the FPD 5, injection device 3 and X-ray source 2 operate in mutual linking. In this embodiment, the manner of judging whether the communication among the devices is possible or not is not limited thereto; whether or not the communication is possible may be judged on the FPD 5, injection device 3 and X-ray source 2 side, and the judgment result may be transmitted to the control unit 15 of the X-ray imaging apparatus 1.

In this embodiment, control in the linking mode is judged to be possible only when all of the following conditions are satisfied: the communication unit 12 of the X-ray imaging apparatus 1 is capable of communicating with all of the FPD 5, injection device 3, and X-ray source 2; the X-ray source 2 is in a state of being able to radiate X-rays; the injection device 3 is in a state of being able to inject the contrast agent; and the FPD 5 is in a state of being able to detect X-rays normally. However, it is possible to exclude from the above conditions a condition which is unlikely to be unsatisfied, and to substitute another judgment condition for any of the above conditions.

Moreover, in this embodiment, the control unit 15 of the X-ray imaging apparatus 1 performs judgment as to whether the X-ray imaging apparatus 1 is in a state of being able to communicate with the FPD 5, injection device 3 and X-ray source 2, and whether the injection device 3 and the X-ray source 2 are in a state of being able to operate normally. However, such judgment may be performed on the FPD 5, injection device 3 and X-ray source 2 side, and the judgment result may be transmitted to the control unit 15 of the X-ray imaging apparatus 1.

When the control unit 15 has judged that it is possible to perform imaging in the linking mode, a screen which permits selection of the linking mode is displayed on the selection screen of the input unit 13. In this way, only when the linking mode can be set, the screen which permits selection of the linking mode is displayed, thus preventing setting the linking mode mistakenly, when selection of the linking mode is not allowed. Moreover, the control unit 15 may be configured to allow the display unit 14 to display the selected kind of imaging, imaging region, subject information, and the like.

When it is set that imaging is performed in the linking mode, the control unit 15 controls the X-ray source 2 through the communication unit 12 such that X-rays are radiated under irradiation conditions in accordance with the linking mode. Here, the irradiation conditions for controlling the X-ray source 2 are conditions for controlling the quality or quantity of the X-rays radiated from the X-ray source 2. For example, the control unit 15 stores beforehand a plurality of irradiation conditions in accordance with the linking mode as well as control patterns corresponding to the irradiation conditions, inside the storage unit thereof. The control unit 15 controls the X-ray source 2 in accordance with the control patterns.

The control under the irradiation conditions in accordance with the linking mode may be, for example, control using a time period elapsed from the time of contrast agent injection or a predetermined data value corresponding to the elapsed time period. For example, a certain period of time is required for the contrast agent to reach the target imaging region from the start of the contrast agent injection, and therefore, irradiation timing of radiating X-rays is determined in consideration of the above period of time. Further, the contrast agent is unevenly distributed in the vessel immediately after the start of the contrast agent injection, and therefore, the X-rays are intensely absorbed only in the vessel. Accordingly, to obtain a clear image of the vessel, the control unit 15 performs control of increasing the X-ray dose immediately after the contrast agent injection. That is, for example, in the case of X-ray tube 23, the tube potential or the tube current supplied from the high voltage power supply 22 is increased or the irradiation time period is increased. As the time passes, the contrast agent is infiltrated into the tissues, and the X-rays are weakly absorbed. Thus, after a predetermined time period from the start of the contrast agent injection, the control unit 15 performs control of reducing the X-ray dose to obtain a clear image of the tissues. In contrast, when an imaging mode other than the linking mode is set, the control unit 15 performs control under an irradiation condition in between the above conditions regardless of the time period elapsed from the contrast agent injection.

To take a moving image in the linking mode, the control unit 15 performs control to satisfy the irradiation condition that X-rays are radiated such that the density of the displayed image does not fluctuate much along with the time elapsed from the contrast agent injection. For example, the concentration of the contrast agent in the vessel is very high for a certain period of time immediately after the start of the contrast agent injection. However, the concentration of the contrast agent decreases as the time passes. Thus, if X-rays are radiated under the same irradiation condition regardless of the time elapsed, there is a shortage of X-ray exposure immediately after the start of the contrast agent injection, and, on the contrary, there is an excess of X-ray exposure after a predetermined period of time. Thus, the control unit 15 controls the X-ray radiation dose in accordance with the time elapsed from the contrast agent injection, so that fluctuation in the density of the vessel image is reduced. In contrast, when an imaging mode other than the linking mode is set, the control unit 15 performs control under an irradiation condition in between the above conditions regardless of the time period elapsed from the contrast agent injection.

When the linking mode is set, the control unit 15 may control the X-ray source 2 so that the X-ray source 2 radiates X-rays under irradiation conditions in accordance with the injection amount of the contrast agent, injection speed, injection pattern and the like. For example, the irradiation conditions may be set such that the X-ray radiation dose is increased as the injection amount or the injection speed of the contrast agent is increased, since X-rays are intensely absorbed under such conditions, while the X-ray radiation dose is reduced as the injection amount or the injection speed of the contrast agent is reduced. The control unit 15 performs control in accordance with these irradiation conditions.

When it is set to perform imaging in the linking mode, the control unit 15 controls the image processing unit 11 so that image processing is performed under image processing conditions in accordance with the linking mode. For example, a plurality of image processing conditions and control patterns corresponding to these image processing conditions in accordance with the linking mode are store beforehand in the storage unit of the control unit 15, and the control unit 15 controls the image processing unit 11 in accordance with the control patterns.

The control under the image processing conditions in accordance with the linking mode may be, for example, control using a time period elapsed from the time of contrast agent injection or a data value corresponding to the elapsed time period, when the linking mode is set. For example, the contrast agent is unevenly distributed in the vessel immediately after the start of the contrast agent injection, and therefore, the X-rays are intensely absorbed only in the vessel. Accordingly, to obtain a clear image of the vessel, the control unit 15 controls the image processing unit 11 for image processing. As the time passes, the contrast agent is infiltrated into the tissues, and the X-rays are weakly absorbed. Thus, after a predetermined time period from the start of the contrast agent injection, the control unit 15 controls the image processing unit 11 for image processing to obtain a clear image of the tissues. In contrast, when an imaging mode other than the linking mode is set, the control unit 15 performs control so that image processing in between the above processing is performed, regardless of the time period elapsed from the contrast agent injection.

To take a moving image in the linking mode, the control unit 15 controls the image processing unit 11 so that image processing is performed such that the density of the displayed image does not fluctuate much along with the time elapsed from the contrast agent injection. For example, the concentration of the contrast agent in the vessel is very high immediately after the start of the contrast agent injection. However, the concentration of the contrast agent decreases as the time passes. Therefore, when the image is displayed without any adjustment, it is difficult to recognize the image. Thus, the control unit 11 controls the image processing unit 11 so that the contrast of the image is adjusted in accordance with the time elapsed from the contrast agent injection. In contrast, when an imaging mode other than the linking mode is set, the control unit 15 performs control so that image processing in between the above processing is performed, regardless of the time period elapsed from the contrast agent injection.

When the linking mode is set, the control unit 15 may control the image processing unit 11 so that the image processing unit 11 performs image processing under image processing conditions in accordance with the injection amount of the contrast agent, injection speed, injection pattern and the like. For example, the image processing conditions may be set such that the density of the image is increased as the injection amount or the injection speed of the contrast agent is increased, since X-rays are intensely absorbed under such conditions, while the density of the image is reduced as the injection amount or the injection speed of the contrast agent is reduced. The control unit 15 performs control in accordance with these image processing conditions.

Each of the control unit 15, input unit 13, display unit 14, communication unit 12, image processing unit 11, and FPD 5 are connected through a bus 16.

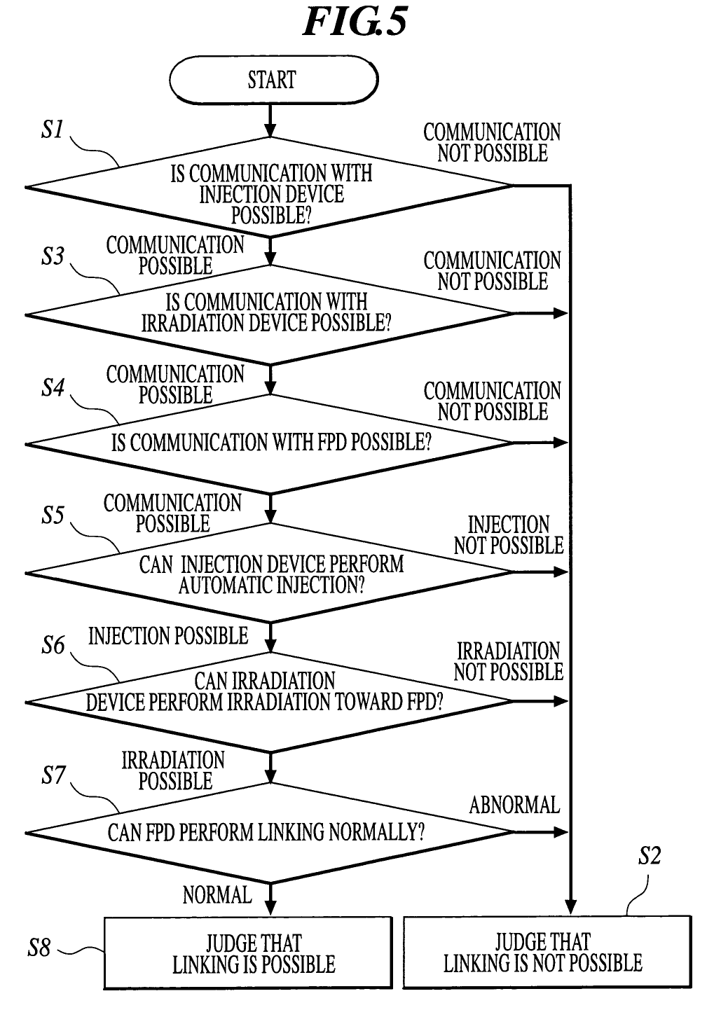

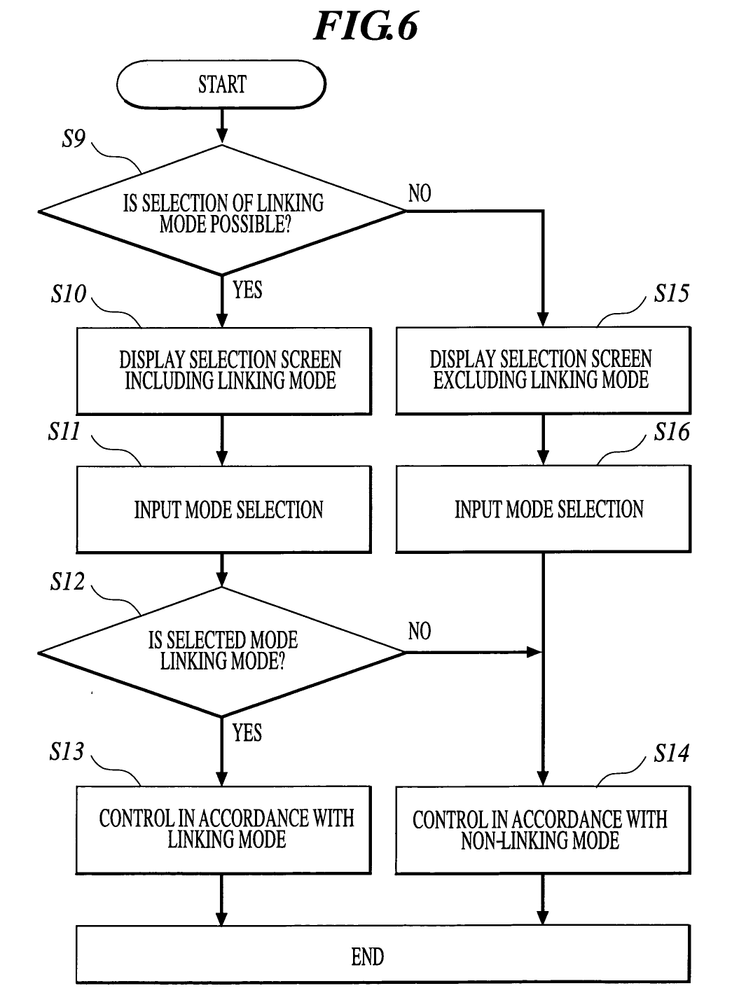

Next, an operation of the X-ray imaging apparatus 1 in this embodiment will be explained with reference to FIGS. 5 and 6.

In this embodiment, the control unit 15 reads the radiographic imaging programs from the storage unit thereof to develop the programs in a predetermined work area. In accordance with the radiographic imaging programs, the control unit 15 controls each unit to perform a series of processing as described below.

First, to perform X-ray imaging, the communication unit 12 of the X-ray imaging apparatus 1 transmits a predetermined signal to the injection device 3, and whether or not the X-ray imaging apparatus 1 and the injection device 3 are in a state of being able to mutually communicate is judged (Step S1). When a predetermined signal is transmitted from the injection device 3 in response to the signal transmitted from the communication unit 12, the control unit 15 judges that the X-ray imaging apparatus 1 and the injection device 3 are in a state of being able to mutually communicate. When there is no response from the injection device 3, the communication unit 15 judges that the X-ray imaging apparatus 1 and the injection device 3 are not in a state of being able to mutually communicate, and that operation in the linking mode is not possible (Step S2). When the control unit 15 has judged that the X-ray imaging apparatus 1 and the injection device 3 are in a state of being able to mutually communicate, a predetermined signal is transmitted from the communication unit 12 of the X-ray imaging apparatus 1 to the X-ray source 2, and whether the X-ray imaging apparatus 1 and X-ray source 2 are in a state of being able to mutually communicate is judged (Step S3). When a predetermined signal is transmitted from the X-ray source 2 in response to the signal transmitted from the communication unit 12, the control unit 15 judges that the X-ray imaging apparatus 1 and the X-ray source 2 are in a state of being able to mutually communicate. When there is no response from the X-ray source 2, the communication unit 15 judges that the X-ray imaging apparatus 1 and the X-ray source 2 are not in a state of being able to mutually communicate, and that operation in the linking mode is not possible (Step S2). When the control unit 15 has judged that the X-ray imaging apparatus 1 and the X-ray source 2 are in a state of being able to mutually communicate, a predetermined signal is transmitted from the communication unit 12 of the X-ray imaging apparatus 1 to the FPD 5, and whether the communication unit 12 of the X-ray imaging apparatus 1 and the FPD 5 are in a state of being able to mutually communicate is judged (Step S4). When a predetermined signal is transmitted from the FPD 5 in response to the signal transmitted from the communication unit 12, the control unit 15 judges that the communication unit 12 and the FPD 5 are in a state of being able to mutually communicate. When there is no response from the FPD 5, the communication unit 15 judges that the communication unit 12 and the FPD 5 are not in a state of being able to mutually communicate, and that operation in the linking mode is not possible (Step S2).

When the control unit 15 has judged that all of the FPD 5, injection device 3, and X-ray source 2 can communicate with the X-ray imaging apparatus 1 through the communication unit 12, the control unit 15 obtains a detection result as to whether or not it is possible for the injection device 3 to perform automatic injection of the contrast agent normally, which is detected by the detection unit provided to the injection device 3, from the detection unit through the communication unit 12. The control unit 15 judges whether it is possible for the injection device 3 to perform automatic injection of the contrast agent normally, based on the obtained detection result (Step S5). When the control unit 15 has judged that the automatic injection is not possible, the control unit 15 judges that operation in the linking mode is not possible (Step S2). On the other hand, when the control unit 15 has judged that the automatic injection is possible, the control unit 15 obtains a detection result as to whether or not it is possible for the X-ray source 2 to radiate X-rays towards the FPD 5 normally, which is detected by the detection unit provided to the X-ray source 2, from the detection unit through the communication unit 12. The control unit 15 judges whether it is possible for the X-ray source 2 to radiate X-rays normally, based on the obtained detection result (Step S6). When the control unit 15 has judged that the X-ray radiation is not possible, the control unit 15 judges that operation in the linking mode is not possible (Step S2). On the other hand, when the control unit 15 has judged that the X-ray radiation is possible, the control unit 15 further judges whether or not it is possible for the FPD 5 to perform reading operation of the X-rays normally, in linking with the injection device 3 and the X-ray source 2 (Step S7). When the control unit 15 has judged that the linked reading operation is not possible, the control unit 15 judges that operation in the linking mode is not possible (Step S2). On the other hand, when the control unit 15 has judged that the linked reading operation is possible, the control unit 15 judges that linking among the FPD 5, injection device 3, and X-ray source 2 is possible (Step S8). The control unit 15 then performs control of each unit in the linking mode.

The order of Steps S1, S3 and S4 is arbitrary, and is not limited to the example described above. As long as Steps S5 and S7 are performed after Steps S1 and S4, respectively, Steps S5, S6 and S7 may be performed in an arbitrary order.

As shown in FIG. 6, the result judged in the flow shown in FIG. 5 is branched in accordance with whether or not it is possible to select as an imaging mode the linking mode where the FPD 5, injection device 3 and X-ray source 2 are mutually linked (Step S9). When the control unit 15 has judged that the selection of the linking mode is possible, a selection screen including the linking mode in the operation panel 131 of the input unit 13 is displayed (Step S10). When an imaging mode is selected and inputted from the input unit 13 by the user (Step S11), the control unit 15 further judges whether or not the imaging mode selected by the user is the linking mode (Step S12). When the selected imaging mode is the linking mode, the control unit 15 performs control on each unit in accordance with the linking mode, such as control of adjusting the X-ray dose radiated from the X-ray source 2 in accordance with the time elapsed from the start of the contrast agent injection from the injection device 3, and control of the X-ray source 2 and the image processing unit 11 so that image processing in the image processing unit 11 is adjusted (Step S13). In contrast, when the selected imaging mode is an imaging mode other than the linking mode, the control unit 15 performs control in the selected imaging mode which is not the linking mode. For example, predetermined control is performed on each unit regardless of the time elapsed from the start of the contrast agent injection (Step S14).

Meanwhile, when the control unit 15 has judged that the selection of the linking mode is not possible, a selection screen excluding the linking mode in the operation panel 131 of the input unit 13 is displayed (Step S15). When an imaging mode is selected and inputted from the input unit 13 by the user (Step S16), the control unit 15 performs control in the selected imaging mode which is not the linking mode. For example, predetermined control is performed on each unit regardless of the time elapsed from the start of the contrast agent injection, in accordance with the inputted imaging mode (Step S14).

As described above, according to the embodiment, the FPD 5, injection device 3 and X-ray source 2, each of which can be used individually, are connected to each other through the network 4 as necessary. For each of the FPD 5, injection device 3 and X-ray source 2, judgment is performed as to whether or not mutual communication is possible and whether or not operation appropriate for the linking thereamong is possible. It is possible to perform control in the linking mode only when the linking is judged as possible. Thus, since each of the FPD 5, injection device 3 and X-ray source 2 can be linked with each other with high accuracy, even when the FPD 5, injection device 3 and X-ray source 2, which are separate devices, are combined for use, X-ray imaging using the contrast agent can be performed without imposing an excessive burden on the subject, as in the case of the all-in-one apparatus.

In the embodiment, a screen permitting selection of the linking mode is displayed on the selection screen of the input unit 13 only when the control unit 15 has judged that the selection of the linking mode is possible. However, regardless of whether or not the selection of the linking mode is possible, a screen permitting the selection of any modes including the linking mode may be displayed on the display screen of the input unit 13, and a message or the like which shows that the selection of the linking mode is not possible may be displayed when the user has selected the linking mode from the input unit 13 when the selection of the linking mode is not possible.

Moreover, the flow of processing of judging whether or not the selection of the linking mode is possible is not limited to the flow shown in FIG. 5, and the judgment may be performed in the order different from that of the flow in FIG. 5.

Further, in the embodiment, the control unit 15 serves as both the judgment unit to judge whether the linking is possible (linking possibility judgment unit) and the control unit of the present invention. However, the linking possibility judgment unit and the control unit of the invention may be different units. Furthermore, in the embodiment, the control unit 15 and the image processing unit 11 are different units; however, a computer may serve as both the control unit 15 and the image processing unit 11.

Thus, it goes without saying that one unit may serve as a plurality of units of the present invention, and one unit of the invention may include a plurality of sections. Further, each of the plurality of units of the invention may include a plurality of sections, and part of the sections may be shared by different units.

Needless to say, the present invention is not limited to the above embodiment.

The entire disclosure of Japanese Patent Application No. 2004-113628 filed on Apr. 7, 2004 including specification, claims, drawings and abstract is incorporated herein by reference in its entirety.

1. A radiographic imaging apparatus comprising:
a plane detection device to obtain radiographic image information by using a plurality of solid-state photodetectors arranged two-dimensionally;
an injection device communication unit to perform communication with an injection device which automatically injects a contrast agent;
an irradiation device communication unit to perform communication with an irradiation device which performs irradiation;
an input unit;
a control unit which includes a plurality of control modes including a linking mode of allowing, by using the injection device communication unit and the irradiation device communication unit, linking among a timing of injecting the contrast agent by the injection device, a timing of obtaining a radiographic image from the plane detection device and a timing of performing the irradiation by the irradiation device, the control unit controlling the injection device, the plane detection device and the irradiation device in a control mode selected from the plurality of control modes in accordance with the input performed through the input unit; and
a judgment unit to judge whether or not control in the linking mode by the control unit is possible,
wherein selection of the linking mode as the control mode is possible only when the judgment unit has judged that the control in the linking mode is possible.

2. The radiographic imaging apparatus of claim 1, wherein a single unit serves as both at least a part of the control unit and at least a part of the judgment unit.

3. The radiographic imaging apparatus of claim 1, wherein selection of a control mode other than the linking mode is also possible, and, when the control mode other than the linking mode is selected, the control unit does not link at least one of the timing of injecting the contrast agent by the injection device, the timing of obtaining the radiographic image from the plane detection device and the timing of performing the irradiation by the irradiation device, with the others.

4. The radiographic imaging apparatus of claim 1, wherein detection as to whether or not the communication between the irradiation device communication unit and the irradiation device is possible is performed, and the judgment unit judges whether or not the control in the linking mode by the control unit is possible based on a result of the detection.

5. The radiographic imaging apparatus of claim 1, wherein detection as to whether or not irradiation toward the plane detection device by the irradiation device is possible is performed, and the judgment unit judges whether or not the control in the linking mode by the control unit is possible based on a result of the detection.

6. The radiographic imaging apparatus of claim 1, wherein detection as to whether or not the communication between the injection device communication unit and the injection device is possible is performed, and the judgment unit judges whether or not the control in the linking mode by the control unit is possible based on a result of the detection.

7. The radiographic imaging apparatus of claim 1, wherein the control unit is permitted to control the plane detection device through wireless communication, the apparatus further comprises a wireless communication state detection unit to detect a state of the wireless communication, and the judgment unit judges whether or not the control in the linking mode by the control unit is possible based on a result of the detection by the wireless communication state detection unit.

8. The radiographic imaging apparatus of claim 1, wherein detection as to whether or not automatic injection of the contrast agent by the injection device is possible is performed, and the judgment unit judges whether or not the control in the linking mode by the control unit is possible based on a result of the detection.

9. The radiographic imaging apparatus of claim 1, further comprising an image processing unit to perform image processing on a radiographic image obtained by the plane detection device,
wherein, when the linking mode is selected, the control unit controls the image processing unit to perform the image processing based on an image processing condition in accordance with the linking mode, and
when a control mode other than the linking mode is selected, the control unit controls the image processing unit to perform the image processing based on an image processing condition in accordance with the selected control mode other than the linking mode.

10. The radiographic imaging apparatus of claim 9, wherein a single unit serves as both at least a part of the control unit and at least a part of the image processing unit.

11. The radiographic imaging apparatus of claim 1, wherein, when the linking mode is selected, the irradiation device communication unit performs the communication to set a condition for controlling the irradiation device in accordance with the linking mode, and
when a control mode other than the linking mode is selected, the irradiation device communication unit performs the communication to set a condition for controlling the irradiation device in accordance with the selected control mode other than the linking mode.

12. The radiographic imaging apparatus of claim 1, wherein, when the linking mode is selected, the irradiation device communication unit performs the communication so that the irradiation device performs the irradiation after a predetermined time period from the timing of injecting the contrast agent by the injection device.

13. The radiographic imaging apparatus of claim 1, wherein, when the linking mode is selected, the control unit controls the plane detection device to read a radiographic image from the plane detection device after a predetermined irradiation reading time period from the timing of performing the irradiation by the irradiation device.

14. The radiographic imaging apparatus of claim 13, wherein the control unit is permitted to control the plane detection unit through wireless communication, and the control unit performs wireless transmission of information concerning a timing of reading the radiographic image, the information being transmitted to the plane detection device prior to radiographic imaging.

15. The radiographic imaging apparatus of claim 1, further comprising an operation screen displaying unit to display an operation screen allowing selection of the linking mode when the judgment unit has judged that the control in the linking mode is possible, and display an operation screen not allowing selection of the linking mode when the judgment unit has judged that the control in the linking mode is not possible.

16. The radiographic imaging apparatus of claim 1, further comprising an operation screen displaying unit to display an operation screen showing an option for selection of the linking mode,
wherein, in a case where the selection of the linking mode is performed through the input unit, the selection of the linking mode is made when the judgment unit has judged that the control in the linking mode is possible, and the selection of the linking mode is not made when the judgment unit has judged that the control in the linking mode is not possible, by displaying on the operation screen that the selection of the linking mode is not possible.

17. The radiographic imaging apparatus of claim 1, wherein the plane detection device is attachable and detachable, when the plane detection device is attached, the plane detection device is adapted to be attached to a radiographing device such that the plane detection device is in a position toward which the irradiation by the irradiation device is performed, the irradiation device is accommodated in the radiographing device, and the plane detection device is a portable FPD of a cassette type.

18. A radiographic imaging program allowing a computer connected to and capable of communicating with a plane detection device to obtain radiographic image information, an injection device to perform automatic injection of a contrast agent, and an irradiation device to perform irradiation, to realize:
a control function of controlling the injection device, the plane detection device and the irradiation device in a control mode selected from a plurality of control modes in accordance with input performed through an input unit, the plurality of control modes including a linking mode of allowing linking among a timing of injecting the contrast agent by the injection device, a timing of obtaining a radiographic image from the plane detection device and a timing of performing the irradiation by the irradiation device;
a judgment function of judging whether or not control in the linking mode is possible; and
a selection possibility determining function of allowing selection of the linking mode as the control mode only when the judgment function has judged that the control in the linking mode is possible.

19. The radiographic imaging program of claim 18, wherein the computer is permitted to control the plane detection device through wireless communication, the program allowing the computer to realize:
a wireless communication state detection function of detecting a state of the wireless communication; and
a judgment function of judging whether or not the control in the linking mode by the control unit is possible based on a result of the detection by the wireless communication state detection function.

20. A computer-readable information storage medium storing the radiographic imaging program of claim 18.
